(19) United States
(12) Patent Application Publication
(10) Pub. No.: US 2025/0248940 A1
(43) Pub. Date: Aug. 7, 2025

(54) Biopolymer Formulations for Drug Delivery

(71) Applicant: 11584022 Canada Inc., Montreal (CA)

(72) Inventors: Thomas DI NARDO, Montreal (CA); George DECKNER, Cincinnati, OH (US)

(73) Assignee: 11584022 Canada Inc., Montreal, QC (CA)

(21) Appl. No.: 18/878,991

(22) PCT Filed: Jun. 29, 2023
(86) PCT No.: PCT/IB2023/056719

§ 371 Date: Dec. 26, 2024

Related U.S. Application Data
(60) Provisional application No. 63/356,703, filed on Jun. 29, 2022.

(51) Int. Cl. A61K 9/16; A61K 8/04; A61K 8/64; A61K 8/65; A61K 8/73; A61K 9/10; A61K 31/167; A61K 31/616; A61Q 19/00

(52) U.S. Cl. CPC A61K 9/1658; A61K 8/044; A61K 8/64; A61K 8/65; A61K 8/731; A61K 8/733; A61K 9/10; A61K 9/1652; A61K 31/167; A61K 31/616; A61Q 19/00

(57) ABSTRACT

Described herein are formulations comprising homogeneous suspensions of biopolymer(s) for drug delivery of active molecules. In embodiments the biopolymer is selected from chitin, chitosan, cellulose, hemicellulose, lignin, amylose, actin, fibrin, collagen, silk, fibroin, keratin, wool, alginic acid and mixtures thereof. In embodiments the biopolymer has been mechanically processed together the API under high-shearing conditions and/or high mechanical energy. The formulations may find particular uses in drug delivery systems, including carrying and releasing a compound or molecule of interest such as a drug product, a drug substance, a cosmetic product, and the like.

### CROSS REFERENCE TO RELATED APPLICATION

The present application claims priority to provisional patent application U.S. 63/356,703 (filed Jun. 29, 2022) the content of which is incorporated herein in its entirety.

### FIELD OF THE INVENTION

The invention relates to the fields of therapeutics and cosmetics, and more particularly to new formulations comprising homogeneous suspensions of biopolymer(s) for drug delivery of active molecules.

### BACKGROUND OF THE INVENTION

Drug delivery systems (DDS) are used to control the rate at which a drug is released and the location in the body where it is released. In this regard, there is a constant need for new approaches, formulations, manufacturing techniques, storage systems, and technologies that can better transport a pharmaceutical compound to its target site to achieve a desired therapeutic effect.

Natural polymers, or biopolymers, have gained popularity as carriers for drug delivery since they offer a plethora of advantages over conventional polymers. Biopolymers are non-toxic and biocompatible, thus making them a versatile carrier. They offer the economic advantage as they are relatively cheap. Biopolymers offer environmental advantages as well because they are biodegradable. However, most abundant biopolymers such as cellulose and chitin are insoluble, thereby limiting and complicating their use. Advantageously, Applicant has described in WO 2022/137184 new homogeneous suspensions of biopolymers that are homogeneous and stable and which may find numerous applications in DDS.

Accordingly, there is still a need for new ways to include biopolymers is DDS.

Also, there is still a need for drug delivery compositions comprising natural, biocompatible, biodegradable and non-toxic ingredients such as cellulose, chitin and the like.

The present invention addresses these needs and other needs as it will be apparent from the review of the disclosure and description of the features of the invention hereinafter.

### BRIEF SUMMARY OF THE INVENTION

According to one aspect, the invention relates to a composition for delivery of an active pharmaceutical ingredient (API), comprising: a biopolymer, and at least one API.

According to another aspect, the invention relates to a method for delivery of an active pharmaceutical ingredient (API) to a subject in need thereof, comprising: (i) providing a composition for delivery as defined herein; and (ii) administering the composition to the subject.

According to another aspect, the invention relates to a method for topical delivery of an active pharmaceutical ingredient (API) to a subject in need thereof, comprising: (i) providing a composition for delivery comprising a biopolymer and at least one API as defined herein; and (ii) applying said composition on the body of said subject.

According to another aspect, the invention relates to a method for oral delivery of an active pharmaceutical ingredient (API) to a subject in need thereof, comprising: (i) providing a mixture comprising an API mixed in a biopolymer composition; (ii) drying the mixture to obtain a dried product; and (iii) orally administering the dried product to the subject.

According to another aspect, the invention relates to a method for manufacturing a composition for delivery as defined herein, the method comprising mechanically processing together the API with biopolymer(s) under high-shearing conditions and/or high mechanical energy to obtain a stable homogeneous aqueous composition.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

For the invention to be readily understood, embodiments of the invention are illustrated by way of example in the accompanying figures.

FIG. 1 is a line graph of time release of acetaminophen from chitin in accordance with Example 1.

FIG. 2 is a line graph of time release of acetaminophen from chitosan, in accordance with Example 2.

FIG. 3 is a line graph of time release of acetyl salicylic acid from chitosan, in accordance with Example 3.

Further details of the invention and its advantages will be apparent from the detailed description included below.

### DETAILED DESCRIPTION OF EMBODIMENTS

In the following description of the embodiments, references to the accompanying figures are illustrations of an example by which the invention may be practised. It will be understood that other embodiments may be made without departing from the scope of the invention disclosed. Unless defined otherwise, all technical and scientific terms used herein have the same meaning as commonly understood by one of ordinary skill in the art to which the invention belongs.

### General Overview

In PCT publication WO 2022/137184 entitled “HOMOGENEOUS BIOPOLYMER SUSPENSIONS, PROCESSES FOR MAKING SAME AND USES THEREOF” (the content of which is incorporated herein by reference in its entirety) Applicant has described the preparation of stable homogeneous suspensions of insoluble and/or semi-soluble biopolymers.

The present inventor(s) have shown that biopolymer suspensions in accordance with present invention have the ability to retain hydrophilic and hydrophobic molecules, making these suspensions useful in drug delivery systems (DDS) as a drug delivery vehicle for transporting and releasing molecules of interest such as active pharmaceutical ingredients (API) and/or other molecule(s). The biopolymer suspensions of the invention may be especially useful when considering the minimal ingredients needed in the suspension, thereby reducing the possibility of undesirable interactions between the vehicle (i.e., suspensions components) and the API. The present invention encompasses, but is not limited to, general and specific compositions described in WO 2022/137184.

Accordingly, the present invention generally relates to the uses of homogeneous suspensions of biopolymers for drug delivery. As used herein the term “drug delivery” generally refers to carrying and releasing a compound or molecule of interest such as a drug product, a drug substance, a cosmetic product, etc. In the present text the terms “composition(s) for drug delivery” and “drug delivery composition(s)”, are used interchangeably.

### Biopolymer

The compositions for drug delivery in accordance with the present invention minimally require a biopolymer. As used herein, the term “biopolymer” refers to natural polymers produced by the cells of living organisms. Biopolymers consist of monomeric units that are covalently bonded to form larger molecules. The present invention encompasses polypeptides, polysaccharides and polynucleotides biopolymers. Other examples of biopolymers include natural rubbers (polymers of isoprene), suberin and lignin (complex polyphenolic polymers), cutin and cutan (complex polymers of long-chain fatty acids) and melanin. In embodiments the biopolymers used as starting materials and obtained in the suspensions are substantially pure, i.e., they consist of only purified natural polymers.

Preferably, the biopolymer used in the drug delivery composition is substantially free from chemical residues and any of such chemical residue is absent or present in undetectable or trace amounts. As used herein, “substantially free from chemical residues” means that chemical compounds, such as acids, bases, reactive chemicals, organic salts and/or inorganic salts, surfactants, dispersing agents (e.g., Tween 80™), a silanizing reagent, acrylamide, etc. are totally absent or merely present in undetectable or trace amounts in the final composition or final suspension. In embodiments, the biopolymer(s) will constitute at least 98%, or at least 99% or at least 99.9% or at least 99.99% by weight of the organic compounds in the biopolymer composition or suspension, i.e., the biopolymer composition or suspension will contain less than 2% or less than 1%, less than 0.1%, or less than 0.01%, or less than 0.001% by weight of organic components other than the biopolymer(s) or degradation product(s).

In embodiments the biopolymer comprises, or alternatively consists of, biopolymer molecules that have been mechanically processed into a stable homogeneous aqueous suspension (e.g., a suspension of insoluble and/or semi-soluble biopolymer particles stably dispersed within a polar solvent).

In accordance with the present invention, the biopolymer may be insoluble or semi-soluble in water. As used herein, the term “insoluble biopolymer” refers to a biopolymer that is “insoluble” in a polar solvent (particularly water) and this term encompasses equivalent terms such as “non-water-soluble”, or “not soluble in water”, or “water-insoluble” or “indissoluble”. Insolubility can typically be observed by a separation, i.e., two separate phases in an aqueous mixture, for instance biopolymer deposits/sediments at a bottom or floating at the top of the aqueous mixture. In accordance with the present invention, examples of insoluble biopolymers include, but are not limited to, chitin, chitosan, cellulose, hemicellulose, lignin, amylose, actin, fibrin, collagen, silk, fibroin, keratin, wool, alginic acid and mixtures thereof. As used herein, the term “semi-soluble biopolymer” refers to a biopolymer that may be solubilized in a polar solvent such as water, but under certain conditions (e.g., molecular weight, heat, addition of chemicals such as acids, alcohols, surfactants, etc.). In accordance with the present invention, examples of semi-soluble biopolymers include, but are not limited to gelatin, pectin, starch, amylopectin, agarose, hyaluronic acid, RNA, DNA, xanthan gum, latex, polymannans, suberin, cutin, cutan, and mixtures thereof.

In embodiments the insoluble biopolymer is selected from chitin, chitosan, cellulose, hemicellulose, lignin, amylose, actin, fibrin, collagen, silk, fibroin, keratin, wool, and mixtures thereof. In embodiments the semi-soluble biopolymer is selected from gelatin, pectin, starch, amylopectin, agarose, alginic acid, alginate, hyaluronic acid, RNA, DNA, xanthan gum, guar gum, carrageenan, latex, polymannans, suberin, cutin, cutan, and mixtures thereof.

Various sources of biopolymer may be used and the present invention is not limited to particular sources of materials. For instance, suitable sources of chitin may include, but are not limited to, green plants, algae, and fungi. Suitable sources of chitin and chitosan may include, but are limited to, fungi, crustaceans (e.g. crabs and shrimps) and insects. In embodiments the insoluble or semi-soluble biopolymer is obtained from fungi and mushrooms. In embodiments the insoluble or semi-soluble biopolymer is obtained from plant materials including, but not limited to, roots, tubers, leaves, petals, seeds, fruits, etc. Preferred sources are non-animal sources.

In embodiments the biopolymer is a 100% natural biopolymer such as SunSpheres Bio™ (microcrystalline cellulose, Dow Chemical), Chemjac™ (amorphophallus konjac root extract and xanthan gum, Chemspire), Kelset™ (sodium alginate, Dupont), Instant Pure-Flo F™ (corn starch, Ingredion), Gelcarin™ GP 379 (carrageenan, iota form, Dupont), Betafib™ MCF (cellulose (and) water-microfibrillated cellulose, Cosun Biobased Products), Betafib™ ETD (cellulose and cellulose gum-microfibrillated cellulose, Cosun Biobased Products), Exilva™ FM02-V,L (cellulose-microfibrillated cellulose, Borregard), Naturesoft™ 800 (cellulose-micro powders), Kelcogel™ CG-HA (gellan gum-CP, Kelco), agar agar, and agarose, mushroom chitosan (e.g., GBS003, Qingdao Chibio Biotech), fungal chitosan (e.g., GBS010, Qingdao Chibio Biotech; or from Kraeber & Co), a fungal derived chitosan (e.g., Kiosmetine-CS™, Kitozyme).

In embodiments the biopolymer is a natural derived biopolymer such as Natrathix™ bio cellulose (cellulose gum, Ashland), Aquasorb™ A500 (cellulose gum, Ashland), Polysurf™ CS 67/Natrosol™ CS plus 330 (cetyl hydroxyethylcellose, Ashland), Structure XL™ (hydroxy propyl starch phosphate, Nouryon), CD-58 (chitosan succinimide, Onlystar Bio-Technology Ltd), carboxymethyl chitosan derivative (GBS010, Qingdao Chibio Biotech), Makimousse™ 7/400 (sodium polyacrylate starch-kobo products, Daito Kasei Kogyo), Salanjul/Sanfresh™ 1000/300sp (sodium polyacrylate starch, Iwase Cosfa USA Inc./Sanyo), Antaron ECoT ethylcellulose (ethyl cellulose, Ashland).

In embodiments the biopolymer comprises a synergistic biopolymer combination, such as Chemjac™ (amorphophallus konjac root extract and xanthan gum, Chemspire), PemuPur™ start (microcrystalline cellulose (and) sphingomonas ferment extract (and) cellulose gum, Lubrizol), Nomcort CG (xanthan gum, ceratonia siliqua gum, Ikeda).

The biopolymer may also comprise other biopolymer actives such as B-CAN™ 55% (oat beta glucan, Adams Food Ingredients) and/or mushroom derived beta glucan.

The present invention encompasses mixtures of two, three, four, five or more insoluble biopolymers including, but not limited to, chitin+chitosan, chitin+ cellulose, chitin+collagen, chitin+silk, chitosan+silk, chitosan+ cellulose, chitosan+collagen, cellulose+collagen, cellulose+silk, collagen+silk, etc. The present invention also encompasses mixtures of two, three, four, five or more semi-soluble biopolymers including, but not limited to agarose+DNA, xanthan gum+starch, latex+alginate, xanthan gum+DNA, guar gum+cutan, etc. It may also be envisioned to mix together two, three, four, five or more insoluble and semi-soluble biopolymers including but not limited to chitin+agarose, chitosan+agarose, chitin+gelatin, chitin+xanthan gum, chitosan+xanthan gum, chitin+sodium hyaluronate, chitosan+sodium hyaluronate, cellulose+sodium hyaluronate, chitin+agarose, chitosan+agarose, cellulose+agarose.

The present invention encompasses also combinations of hydrophobically modified biopolymers and unmodified biopolymers which can form stable, viscous oil in water emulsions when dispersed using high shear processes and/or mechanical energy (with or without emulsifiers), Examples of hydrophobically modified biopolymers include Natrosol™ CS Plus 330/Polysurf™ CS 67 (cetyl hydroxyethylcellose, Ashland), StarDesign Ultra™ (sodium starch octenylsuccinate, Cargill Beauty), Inutec™ SP1 (inulin lauryl carbamate, Beneo), Texturlux Stabil™ (hydrolyzed corn starch hydroxyethyl ether, Primient).

In embodiments, the biopolymer consists of a biopolymer composition comprising biopolymer molecules that have been mechanically processed into a stable homogeneous aqueous biopolymer suspension. As used herein the term “homogeneous” generally refers to the appearance of the suspension under the naked eye (e.g., uniform color, uniform texture, etc.). Homogenous as used herein does not exclude the possibility that the suspension is “heterogenous” at the molecular level (e.g., various particles size, presence of aggregates, etc.). As used herein, the terms “stable homogeneous aqueous biopolymer suspension”, or similar terms that may be used herein interchangeably such as “homogenous biopolymer suspension” or “stable biopolymer suspension” or simply “biopolymer suspension”, all refer to a suspension of insoluble and/or semi-soluble biopolymer particles that have been stably dispersed within a polar solvent. The polar solvent may be a polar protic solvent or a polar aprotic solvent. The polar solvent may be an aqueous solvent. The insoluble and/or semi-soluble biopolymer particles that are present in biopolymer suspensions may be shaped like fibers and/or like agglomerated spheres or agglomerated bodies. Stability of the biopolymer suspensions may be assessed by any suitable means. In preferred embodiments, the stability is measured or observed by a lack of separation, i.e., one single phase instead of two separate phases in an aqueous mixture, for instance absence of biopolymer deposits/sediments at a bottom or floating at the top of the aqueous mixture. Preferably, biopolymer suspensions in accordance with the present invention are stable (e.g. absence of separation) for at least 1 day, or at least 1 week, or at least one month, or at least one year or more.

Notwithstanding the above, those skilled in the art understand that insoluble and/or semi-soluble biopolymers may never become truly soluble. Instead, they become “swellable” and bind water which is why the biopolymer becomes a viscous suspension during the high-shearing conditions and/or mechanical energy to which the biopolymer(s) are submitted in accordance with the present invention. Accordingly, the present invention encompasses both, “swellable biopolymers” as well as “non-swellable biopolymers” since a non swellable polymer could be swellable using high-shearing and/or mechanical energy. As used herein, swellable biopolymers encompasses biopolymers that absorb and bind water, which results in an increase in their particle size and water dispersion viscosity.

In embodiments, the biopolymer is swellable with wet ball milling. This may include, but it is not limited to, chitin, chitosan, hemicellulose and pregelatinized corn starch.

In embodiments, the biopolymer is swellable with high shear processes other than ball milling. This may include, but it is not limited to, microcrystalline cellulose, microfibrillated cellulose, nano cellulose, hairy nanocellulose, konjac glucomannan, hydroxypropyl starch phosphate, high acyl gellan gum, gellan gum, carboxymethyl starch, carboxymethyl cellulose (low ds type), agar agar, and agarose.

It is also conceivable in accordance with the present invention to use soluble biopolymers including, but not limited to, xanthan gum, diutan gum, sodium alginate, sclerotium gum.

In embodiments, the biopolymer molecules or particles that are part of the compositions for drug delivery have been mechanically processed into a stable homogeneous aqueous biopolymer suspension. In embodiments the mechanical processing involves high-shearing conditions and/or high mechanical energy. In embodiments the high-shearing conditions and/or high mechanical energy is obtained by a process including, but not limited to mechanical shearing, sheer thinning, planetary ball milling, rolling mill, vibrating ball mill, tumbling stirred ball mill, horizontal media mill, colloid milling. As indicated hereinafter, the high-shearing conditions and/or high mechanical energy can be carried out for a duration, under parameters, under suitable conditions, etc. until a desirable change of state is obtained, e.g., change of color, a change in viscosity, a change from a slurry to a paste, ointment, cream, lotion, gel or milk, etc.

Without wishing to be bound by theory, submitting the biopolymer to high-shearing conditions and/or high mechanical energy improves biopolymer performance not seen using conventional processes, including improved rheological properties such as viscosity, shear thinning properties, and high yield value. biopolymers dispersed using high shear processes may contain lamellar crystalline gel networks (LGN) that may synergistically increase the viscosity of the biopolymer dispersions. Oil+water biopolymer dispersions in accordance with the present invention may also contain Pickering emulsion wherein the water-in-oil or oil-in-water emulsion is stabilized by the biopolymer.

In embodiments the high-shearing conditions and/or high mechanical energy requires using a suitable device or apparatus including, but not limited to, ball miller (e.g., planetary ball miller, rolling miller, vibrating ball miller, tumbling stirred ball miller, horizontal media mill, colloid miller, a magnetic miller), a twin-screw extruder, a high-pressure homogenizer, a blade homogenizer, a stirring homogenizer, a disperser, a rotor-stator homogenizer, a high-shear mixer, a plowshare mixer, a dynamic mixer, a plough mixer, a turbine mixer, a speed mixer, an attrition miller, a sonicator (e.g. high shear ultrasonic processes), a tissue tearor, a cell lysor, a polytron, a ribbon agitator, a microfluidizer, a high pressure homogenizer, and combinations thereof. In preferred embodiments, the present invention utilizes ball milling under wet conditions. Particular examples of ball miller include, but are not limited to, vertical planetary mill (e.g., Tencan XQM-2 A™) with 100 ml capacity zirconia jars and 10 mm diameter zirconia balls, Flacktek™ speed mixer (DAC 330-11 SE) with 40 mL zirconia jar with 5 mm diameter zirconia balls or zirconia rings, 1.5 L Supermill Plus™ with 1.4-1.7 mm zirconia beads and Netzsch mill Labstar™ with 0.6-0.8 mm beads or 1.4-1.7 mm beads.

In particular embodiments, biopolymer compositions and suspensions in accordance with the present invention are obtained using a particular protocol referred herein as the “10+1 Alt method”. This method comprises milling of the biopolymer for a certain period of time (e.g., 10 min) followed by a short pause (e.g., 1 min) then milling in the opposite direction for a certain period of time (e.g., 10 min) for a total of 1 hour, or 2 hours, or 3 hours, or 5 hours, 10 hours, or 12 hours.

Advantageously, the viscosity of the compositions/suspensions can be altered by varying the high-shearing conditions and/or mechanical energy to which the biopolymer(s) are submitted. These conditions can be adjusted to obtain a stable homogeneous suspension (e.g., a stable colloidal homogeneous suspension) having a desired viscosity. Typically, providing more mechanical energy will increase the shearing and will reduce accordingly the viscosity of the end product. The biopolymer itself and/or the final compositions for drug delivery may be formulated as a paste, an ointment, a cream, a lotion, a gel or a milk.

In embodiments, the biopolymer, and/or the biopolymer compositions comprising biopolymer molecules or particles, is a colloidal homogeneous biopolymer suspension. In embodiments, the colloidal homogeneous suspension comprises colloids having a range from about 1 nm to about 1 μm.

In embodiments, the biopolymer, and/or the biopolymer compositions comprising biopolymer molecules, comprises biopolymer fibers. In embodiments the fibers have of a width of about 1 nm to about 5 μm, or about 5 nm to about 5 μm, about 7 nm to about 5 μm, or about 10 nm to about 5 μm, or about 20 nm to about 5 μm, or about 25 nm to about 5 μm, or about 30 nm to about 5 μm, or about 35 nm to about 5 μm, or about 35 nm to about 3 μm. In embodiments the fibers having of a width of at least 1 nm, or at least 5 nm, or at least 10 nm, or at least 20 nm, or at least 30 nm, or at least 40 nm, or at least 50 nm, or at least 75 nm, or at least 100 nm, or at least 250 nm, or at least 500 nm, or at least 750 nm, or at least 1 μm, or at least 2 μm, or at least 3 μm, or at least 4 μm, or at least 5 μm, or wider.

In embodiments the biopolymer, and/or the biopolymer compositions comprising biopolymer molecules, comprises biopolymer fibers having a length of about 1 nm to about 200 μm, of about 10 nm to about 100 μm, or about 50 nm to about 10 μm, or about 100 nm to about 10 μm, or about 500 nm to about 10 μm, or about 750 nm to about 10 μm, or about 800 nm to about 10 μm, or about 900 nm to about 5 μm, or about 1 μm to about 10 μm, or about 1 μm to about 5 μm, or about 1 μm to about 3 μm. In embodiments the fibers have of a length of at least 1 nm, or at least 10 nm, at least 50 nm, or at least 100 nm, or at least 250 nm or at least 500 nm, or at least 750 nm, or at least 800 nm, or at least about 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer. In embodiments, a dry particle size range may be between about 1 nm to about 1 μm, or up to 10 μm, and a wet particle size range may be between about 200 nm to about 20 μm, or up to 200 μm.

In embodiments the biopolymer, and/or the biopolymer compositions comprising biopolymer molecules, comprises biopolymer fibers having both: (i) a width greater than 20 nm (e.g., at least 25 nm, or at least 40 nm, or at least 50 nm,) and a length greater than 50 nm (e.g., at least 100 nm, or at least 500 nm, or at least 1 μm, or at least 2 μm); or (ii) a width greater than 32 nm (e.g., at least 35 nm, or at least 40 nm, or least 50 nm) and a length of than 50 nm (e.g., at least 100 nm, or at least 500 nm, or at least 1 μm, or at least 2 μm); or (iii) a width greater than 20 nm (e.g., at least 25 nm, or at least 40 nm, or least 50 nm) and a length of than 500 nm (e.g., at least 600 nm, or at least 750 nm, or at least 1 μm, or at least 2 μm); or (iv) a width greater than 30 nm (e.g., at least 35 nm, or at least 40 nm, or least 50 nm) and a length of than 800 nm (e.g., at least 900 nm, or at least 1 μm, or at least 2 μm); or (v) a width greater than 8 nm (e.g., at least 10 nm, at least 25 nm, or at least 35 nm, or at least 40 nm, or least 50 nm) and a length of than 340 nm (e.g., at least 350 nm, or at least 500 nm, at least 750 nm, or at least 900 nm, or at least 1 μm, or at least 2 μm); or (vi) a width greater than 11 nm (e.g., at least 15 nm, at least 25 nm, or at least 35 nm, or at least 40 nm, or least 50 nm) and a length of than 166 nm (e.g., at least 200 nm, or at least 350 nm, or at least 500 nm, at least 750 nm, or at least 900 nm, or at least 1 μm, or at least 2 μm); or (viii) a width greater than 32 nm (e.g., at least 35 nm, or at least 40 nm, or least 50 nm) and a length greater than 800 nm (e.g., at least 900 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or longer).

In embodiments the biopolymer, and/or the biopolymer compositions comprising biopolymer molecules, comprises biopolymer fibers wherein the average width and average length of the fibers in the composition are as defined hereinabove, e.g. an average width greater than 20 nm (e.g., at least 25 nm, or at least 40 nm, or at least 50 nm) and an average length greater than 50 nm (e.g., at least 60 nm, at least 75 nm, or at least 100 nm, or at least 500 nm, at least 750 nm, or at least 1 μm, or at least 2 μm, or at least 3 μm, or at least 4 μm, or at least 5 μm, or at least 6 μm, or at least 7 μm, or at least 8 μm, or at least 9 μm, or at least 10 μm, or at least 25 μm, or at least 50 μm, or at least 75 μm, or at least 100 μm, or at least 150 μm, or at least 200 μm or wider).

In embodiments the biopolymer suspensions and/or biopolymer compositions comprising biopolymer molecules, have a pH between about 6.5 and about 8.5. In particular embodiments the biopolymer suspension is a chitosan suspension having a pH between about 7.8 and about 8.1.

In embodiments the biopolymer, and/or the biopolymer compositions comprising biopolymer molecules, comprises biopolymer fibers having both a crystalline region and an amorphous region. In embodiments the stable homogeneous suspension comprises biopolymer fibers having a globular shape. In embodiments the stable homogeneous suspension is comprised of mainly, or only, of suspended biopolymer nanofibrils.

Those skilled in the art are aware that particle size measurements may vary according to the measurement method and the state of the particles (e.g., particles in a wet state are typically larger than the same particles in a dry state). Typically, the particles will be in a wet or suspended stage when measured by dynamic light scattering (DLS) and in a dry stage when measured by scanning electron microscopy (SEM).

In embodiments the biopolymer, and/or the biopolymer compositions comprising biopolymer molecules, comprises agglomerated spheres of alginic acid having an average size of about 40 nm to about 80 nm, or about 45 nm to about 75 nm, as measured by scanning electron microscopy (SEM). In embodiments, the stable homogeneous suspension comprises agglomerated spheres of alginic acid having a median size of about 30 nm to about 70 nm or about 35 nm to about 65 nm, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of cellulose having an average size of about 50 nm to about 80 nm, or about 55 nm to about 75 nm, average size of about 40 nm to about 80 nm, or about 45 nm to about 75 nm, as measured by scanning electron microscopy (SEM). In embodiments the stable homogeneous biopolymer suspension comprises agglomerated spheres of cellulose having a median size of about 35 nm to about 75 nm or about 40 nm to about 65, average size of about 40 nm to about 80 nm, or about 45 nm to about 75 nm, as measured by scanning electron microscopy (SEM).

In embodiments the biopolymer, and/or the biopolymer compositions comprising biopolymer molecules, comprises agglomerated spheres of chitin having an average size of about 45 nm to about 85 nm, or about 50 nm to about 80 nm. In embodiments the stable homogeneous biopolymer suspension comprises agglomerated spheres of cellulose having a median size of about 45 nm to about 80 nm or about 50 nm to about 75 nm, as measured by scanning electron microscopy (SEM).

In embodiments the biopolymer, and/or the biopolymer compositions comprising biopolymer molecules, comprises agglomerated spheres of chitosan having an average size of about 75 nm to about 120 nm, or about 80 nm to about 115 nm, or about 85 nm to about 110 nm, as measured by scanning electron microscopy (SEM). In embodiments the stable homogeneous suspension comprises agglomerated spheres of chitosan having a median size of about 70 nm to about 100 nm or about 75 nm to about 95 nm, as measured by scanning electron microscopy (SEM).

In embodiments the biopolymer, and/or the biopolymer compositions comprising biopolymer molecules, comprises agglomerated spheres of silk having an average size of about 40 nm to about 165 nm, or about 45 nm to about 160 nm, as measured by scanning electron microscopy (SEM). In embodiments the stable homogeneous biopolymer suspension comprises agglomerated spheres of silk having a median size of about 40 nm to about 150 nm or about 45 nm to about 140, as measured by scanning electron microscopy (SEM).

In embodiments the biopolymer, and/or the biopolymer compositions comprising biopolymer molecules, comprises particles of one or more of alginic acid, cellulose, chitin, chitosan and silk, wherein the range of particle sizes, as measured by SEM is as defined in the tables and figures of WO 2022/137184.

In embodiments the biopolymer, and/or the biopolymer compositions comprising biopolymer molecules, is (are) characterized by visual properties like those depicted in the SEM images shown in the figures of WO 2022/137184.

In embodiments the biopolymer, and/or the biopolymer compositions comprising biopolymer molecules, is (are) characterized by a Fourier Transform Infrared Spectroscopy (FTIR) spectrum as depicted in the figures of WO 2022/137184.

In embodiments the biopolymer, and/or the biopolymer compositions comprising biopolymer molecules, is (are) characterized by Solid-State Nuclear Magnetic Resonance characterization (SSNMR) as depicted in in the figures of WO 2022/137184.

In embodiments the biopolymer, and/or the biopolymer compositions comprising biopolymer molecules, is (are) characterized by Power X-Ray Diffraction (PXRD) pattern(s) as depicted in the figures of WO 2022/137184.

In embodiments the biopolymer, and/or the biopolymer compositions comprising biopolymer molecules, is (are) characterized by Dynamic Light Scattering (DLS) measurements like those reported in WO 2022/137184.

In embodiments the biopolymer, and/or the biopolymer compositions comprising biopolymer molecules, is (are) characterized by a transmittance spectrum as shown in the figures of WO 2022/137184.

In embodiments the biopolymer, and/or the biopolymer compositions comprising biopolymer molecules, is (are) characterized by a sweep suspension test as reported in WO 2022/137184.

In embodiments the biopolymer, and/or the biopolymer compositions comprising biopolymer molecules, is (are) characterized by a rheological behaviour as depicted in the figure(s) s WO 2022/137184.

### Active Pharmaceutical Ingredients

The present invention aims, among other things, to carry, transport and/or provide a vehicle for carrying, delivering and releasing compounds of interest.

In embodiments, the compound of interest is an “active pharmaceutical ingredient” or “API”. As used herein the term active pharmaceutical ingredient or API encompasses any substance or compound that can provide at least one beneficial health benefit to a subject. For instance, the API may be a cosmetic compound, a pharmaceutical compound, or a drug substance, etc.

As used herein, the term “subject” includes living organisms to delivery of compounds of interest is desired. The term “subject” includes animals (e.g., mammals (e.g., cats, dogs, horses, pigs, cows, goats, sheep, rodents (e.g., mice or rats), rabbits, squirrels, bears, primates (e.g., chimpanzees, monkeys, gorillas, and humans)), as well as avian (e.g. chickens, ducks, Peking ducks, geese), and transgenic species thereof. Preferably, the subject is a human or a non-human primate (e.g., chimpanzee, monkey, macaque, gorilla). More preferably, the subject is a human. Even more preferably the subject is a human in need of treatment and having, or likely to have, one of more undesirable health problems and/or symptoms. In embodiments the subject is a human who may benefit from delivery and release of pharmaceutics of cosmetics, for instance on its skins or layer thereof.

The pharmaceutical compound may be any compound with pharmaceutical activity including, but not limited to, analgesics, anesthetics, anti-addiction agents, antibacterials, anticonvulsants, antidementia agents, antidepressants, antiemetics, antifungals, antigout agents, anti-inflammatoirentimigraine agents, antimyasthenic agents, antimycobacterials, antineoplastics, anti-obesity agents, antiparasitics, antiparkinson agents, antipsychotics, antispasticity agents, antivirals, anxiolytics, bipolar agents, blood glucose regulators, blood products, cardiovascular agents, central nervous system agents, contraceptives, dental and oral agents, dermatological agents, electrolytes, minerals, metals, vitamins, gastrointestinal agents, genitourinary agents, hormonal agents (adrenal), hormonal agents (pituitary), hormonal agents (prostaglandins), hormonal agents (sex hormones), hormonal agents (thyroid), hormone suppressant (adrenal), hormone suppressant (pituitary), hormone suppressant (thyroid), immunological agents, infertility agents, inflammatory bowel disease agents, metabolic bone disease agents, ophthalmic agents, otic agents, respiratory tract agents, sexual disorder agents, skeletal muscle relaxants, sleep disorder agents.

In embodiments, the pharmaceutical compound is selected from acetaminophen, acetyl salicylic acid, acyclovir, acrivastine, adalimumab, alendronic acid, allopurinol, alogliptin, amitriptyline for depression, amitriptyline for pain and migraine, amlodipine, amoxicillin, anastrozole, apixaban, atenolol, atorvastatin, azathioprine, azithromycin, baclofen, beclometasone inhalers, beclometasone nasal spray, beclometasone skin creams, bendroflumethiazide, benzoyl peroxide, benzydamine, betahistine, betamethasone for eyes, ears and nose, betamethasone for skin, bimatoprost, bisacodyl, bisoprolol, brinzolamide, budesonide inhalers, budesonide nasal spray, budesonide rectal foam and enemas, bumetanide, buprenorphine for pain, buscopan (hyoscine butylbromide), candesartan, carbamazepine, carbimazole, carbocisteine, carmellose sodium, carvedilol, cefalexin, cetirizine, varenicline, chloramphenicol, chlorhexidine, chlorphenamine, cinnarizine, ciprofloxacin, citalopram, clarithromycin, clobetasol, clobetasone, clonazepam, clopidogrel, clotrimazole, clotrimazole, co-amoxiclav, co-beneldopa, co-careldopa, co-codamol for adults, co-codamol for children, co-codaprin (aspirin and codeine), co-dydramol, codeine, colchicine, cyanocobalamin, cyclizine, dabigatran, dapagliflozin, dexamethasone tablets and liquid, diazepam, diclofenac, digoxin, dihydrocodeine, diltiazem, diphenhydramine, dipyridamole, docusate, domperidone, donepezil, dosulepin, doxazosin, doxycycline, duloxetine, edoxaban, empagliflozin, enalapril, eplerenone, erythromycin, escitalopram, esomeprazole, ezetimibe, felodipine, fentanyl, ferrous fumarate, ferrous sulfate, fexofenadine, finasteride, flucloxacillin, fluconazole, fluoxetine, fluticasone inhalers, fluticasone nasal spray and drops, fluticasone skin creams, folic acid, furosemide, fusidic acid, fybogel (ispaghula husk), gabapentin, gliclazide, glimepiride, glyceryl trinitrate, heparinoid, hydrocortisone, hydrocortisone buccal tablets, hydrocortisone for piles and itchy bottom, hydrocortisone for skin, hydrocortisone injections, hydrocortisone rectal foam, hydrocortisone tablets, hydroxocobalamin, hyoscine hydrobromide, ibuprofen, indapamide, irbesartan, isosorbide mononitrate, isosorbide dinitrate, isotretinoin capsules, isotretinoin gel, ketoconazole, labetalol, lactulose, lamotrigine, lansoprazole, latanoprost, lercanidipine, letrozole, levetiracetam, levothyroxine, lidocaine, linagliptin, lisinopril, lithium, loperamide, loratadine, lorazepam, losartan, low-dose aspirin, lymecycline, macrogol, mebendazole, mebeverine, melatonin, mesalazine, metformin, methadone, methotrexate, methylphenidate, metoclopramide, metoprolol, metronidazole, mirabegron, mirtazapine, molnupiravir, mometasone, montelukast, morphine, naproxen, nefopam, nicorandil, nifedipine, nitrofurantoin, nortriptyline, nystatin, olanzapine, olmesartan, omeprazole, oxybutynin, oxycodone, pantoprazole, paroxetine, nirmatrelvir, peppermint oil, perindopril, phenoxymethylpenicillin, phenytoin, pioglitazone, pravastatin, prednisolone tablets and liquid, pregabalin, prochlorperazine, promethazine, propranolol, pseudoephedrine, rabeprazole, ramipril, ranitidine, remdesivir, risedronate, risperidone, rivaroxaban, ropinirole, rosuvastatin, salbutamol inhaler, saxagliptin, senna, sertraline, sildenafil, simeticone, simvastatin, sitagliptin, sodium valproate, solifenacin, sotalol, sotrovimab, sulfasalazine, sumatriptan, tadalafil, tamsulosin, temazepam, terbinafine, thiamine (Vitamin B1), ticagrelor, tolterodine, topiramate, tramadol, tranexamic acid, trazodone, trimethoprim, valproic acid, valsartan, varenicline, venlafaxine, verapamil, zolpidem, and zopiclone.

In embodiments, the pharmaceutical compound is a compound that is uses for treating and/or preventing a medical condition selected from: Acne, Acute cholecystitis, Acute lymphoblastic leukaemia, Acute myeloid leukaemia, Acute pancreatitis, Addison's disease, Alcohol-related liver disease, Allergic rhinitis, Allergies, Alzheimer's disease, Anal cancer, Anaphylaxis, Angioedema, Ankylosing spondylitis, Anxiety, Anxiety disorders, Appendicitis, Arthritis, Asthma, Atopic eczema, Attention deficit hyperactivity disorder (ADHD), Autistic spectrum disorder (ASD), Bacterial vaginosis, Benign prostate enlargement, Bile duct cancer (cholangiocarcinoma), Binge eating, Bipolar disorder, Bladder cancer, Blood poisoning (sepsis), Bone cancer, Bowel cancer, Bowel incontinence, Bowel polyps, Brain tumours, Breast cancer, Bronchiectasis, Bronchitis, Bulimia, Bunion, Carcinoid syndrome and carcinoid tumours, Catarrh, Cellulitis, Cervical cancer, Chest infection, Chest pain, Chickenpox, Chilblains, Chlamydia, Chronic fatigue syndrome, Chronic kidney disease, Chronic lymphocytic leukaemia, Chronic myeloid leukaemia, Chronic obstructive pulmonary disease, Chronic pancreatitis, Cirrhosis, Clostridium difficile, Coeliac disease, Cold sore, Coma, Common cold, Common heart conditions, Congenital heart disease, Conjunctivitis, Constipation, Coronavirus (COVID-19), Cough, Crohn's disease, Croup, Cystic fibrosis, Cystitis, Deafblindness, Deep vein thrombosis, Dehydration, Dementia, Dementia with Lewy bodies, Dental abscess, Depression, Dermatitis herpetiformis, Diabetes, Diarrhoea, Discoid eczema, Diverticular disease and diverticulitis, Dizziness (Lightheadedness), Down's syndrome, Dry mouth, Dysphagia (swallowing problems), Dystonia, Earache, Earwax build-up, Ebola virus disease, Ectopic pregnancy, Endometriosis, Epilepsy, Erectile dysfunction (impotence), Escherichia coli (E. coli) O157, Ewing sarcoma, Ewing sarcoma: Children, Eye cancer, Febrile seizures, Fever in adults, Fever in children, Fibroids, Fibromyalgia, Flatulence, Flu, Foetal alcohol syndrome, Food poisoning, Fungal nail infection, Gallbladder cancer, Gallstones, Ganglion cyst, Gastroenteritis, Gastro-oesophageal reflux disease (GORD), Genital herpes, Genital warts, Germ cell tumours, Glandular fever, Gonorrhoea, Gout, Gum disease, Haemorrhoids (piles), Hairy cell leukaemia, Hand, foot and mouth disease, Hay fever, Head and neck cancer, Head lice and nits, Headaches, Hearing loss, Heart failure, Hepatitis A, Hepatitis B, Hepatitis C, Hiatus hernia, High cholesterol, HIV, Hodgkin lymphoma, Hodgkin lymphoma: Children, Hodgkin lymphoma: Teenagers and young adults, Huntington's disease, Hyperglycemia (high blood sugar), Hyperhidrosis, Hypoglycaemia (low blood sugar), Idiopathic pulmonary fibrosis, Impetigo, Indigestion, Ingrown toenail, Inherited heart conditions, Insomnia, Iron deficiency anaemia, Irritable bowel syndrome (IBS), Irritable hip, Itching, Itchy bottom, Kaposi's sarcoma, Kidney cancer, Kidney infection, Kidney stones, Labyrinthitis, Lactose intolerance, Langerhans cell histiocytosis, Laryngeal (larynx) cancer, Laryngitis, Leg cramps, Lichen planus, Liver cancer, Liver disease, Liver tumours, Loss of libido, Lung cancer, Lupus, Lyme disease, Lymphoedema, Lymphogranuloma venereum (LGV), Malaria, Malignant brain tumour (cancerous), Malnutrition, Measles, Meningitis, Menopause, Mesothelioma, Middle ear infection (otitis media), Migraine, Motor neurone disease (MND), Mouth cancer, Mouth ulcer, Multiple myeloma, Multiple sclerosis (MS), Mumps, Meniere's disease, Nasal and sinus cancer, Nasopharyngeal cancer, Neuroblastoma, Neuroendocrine tumours, Non-alcoholic fatty liver disease (NAFLD), Non-Hodgkin lymphoma, Non-Hodgkin lymphoma: Children, Norovirus, Nosebleed, Obesity, Obsessive compulsive disorder (OCD), Obstructive sleep apnoea, Oesophageal cancer, Oral thrush in adults, Osteoarthritis, Osteoporosis, Osteosarcoma, Otitis externa, Ovarian cancer, Ovarian cancer: Teenagers and young adults, Ovarian cyst, Overactive thyroid, Paget's disease of the nipple, Pancreatic cancer, Panic disorder, Parkinson's disease, Pelvic inflammatory disease, Pelvic organ prolapse, Penile cancer, Peripheral neuropathy, Personality disorder, Pleurisy, Pneumonia, Polymyalgia rheumatica, Post-traumatic stress disorder (PTSD), Postnatal depression, Pregnancy and baby, Pressure ulcers, Prostate cancer, Psoriasis, Psoriatic arthritis, Psychosis, Pubic lice, Rare tumours, Raynaud's phenomenon, Reactive arthritis, Restless legs syndrome, Retinoblastoma, Retinoblastoma: Children, Rhabdomyosarcoma, Rheumatoid arthritis, Ringworm and other fungal infections, Rosacea, Scabies, Scarlet fever, Schizophrenia, Scoliosis, Septic shock, Shingles, Shortness of breath, Sickle cell disease, Sinusitis, Sjogren's syndrome, Skin cancer (melanoma), Skin cancer (non-melanoma), Slapped cheek syndrome, Soft tissue sarcomas, Soft tissue sarcomas: Teenagers and young adults, Sore throat, Spleen problems and spleen removal, Stillbirth, Stomach ache and abdominal pain, Stomach cancer, Stomach ulcer, Stress, anxiety and low mood, Stroke, Sudden infant death syndrome (SIDS), Suicide, Sunburn, Swollen glands, Syphilis, Testicular cancer, Testicular cancer: Teenagers and young adults, Testicular lumps and swellings, Thirst, Threadworms, Thrush, Thyroid cancer, Thyroid cancer, Tinnitus, Tonsillitis, Tooth decay, Toothache, Transient ischaemic attack (TIA), Trigeminal neuralgia, Tuberculosis (TB), Type 1 diabetes, Type 2 diabetes, Trichomonas infection, Ulcerative colitis, Underactive thyroid, Urinary incontinence, Urinary tract infection (UTI), Urinary tract infection (UTI) in children, Urticaria (hives), Vaginal cancer, Vaginal thrush, Varicose eczema, Venous leg ulcer, Vertigo, Vitamin B12 or folate deficiency anaemia, Vomiting in adults, Vulval cancer, Warts and verrucas, Whooping cough, Wilms' tumour, Womb (uterus) cancer, Yellow fever

The API may be any compound useful in cosmetics including, but not limited to, a compound for moisturizing the skin, a compound for improving skin appearance, a compound for protecting from environmental damages, a compound for fighting the effects of aging, a compound for removing wrinkles and/or sunspots, and/or a compound for the prevention of any of the following skin conditions: acne, acne/folliculitis, acne scars, aging skin, blemishes, broken blood vessels, brown spots or other discolored skin, cellulite, creases around the mouth or nose, deflated or sinking around cheeks, temples, lips and eyes, dull and discolored skin, excessive facial or body hair, enlarged pores, fatigued or tired appearance, fine lines and creases, flaking and cracking skin, flushed appearance, freckles, furrows or crinkles in the forehead, hair loss, hair removal, hirsutism and hypertrichosis, hyperpigmentation, melasma, rosacea, sagging or loss of volume, scarring of skin, skin cancer, spider veins in the legs and face, sun damaged skin, sunspots, tattoo removal, textural and tone changes, tiny lines near the eyes or mouth, visible facial blood vessels, wrinkles and fine lines, etc.

The API may also be any compound useful in hygiene products such as anti-acne products, toothpaste and anti-cavity products, antiperspirant, astringents, corn and callus removers, dandruff products, hair growth/hair loss products, nail biting products, and wart remover. The API may also be any compound useful in other skin products such as skin bleaching, sunscreen, topical analgesic, topical anti-fungal products and anti-microbial products.

In embodiments, the cosmetic compound is selected from antioxidants (e.g. resveratrol, vitamin C), alpha hydroxy acid (AHA), hyaluronic acid, retinoids (e.g., retinol), vitamins (e.g., vitamin A, vitamin E), resorcinol, benzoyl peroxide, salicylic acid, ceramides, niacinamide, sulfur, sodium fluoride, sodium monophosphate, clioquinol, haloprogin, miconazole nitrate, povidone-iodine, tolnaftate, undecylenic acid, calcium undecylenate, copper undecylenate, zinc undecylenate, clotrimazole bacitracin, chlortetracycline, neomycin sulfate, tetracycline hydrochloride, aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrate, aluminum zirconium tetrachlorohydrex gly, aluminum zirconium trichlorohydrate, aluminum zirconium trichlorohydrex gly, allantoin, aluminum hydroxide gel, calamine, cocoa butter, cod liver oil, dimethicone, glycerin, kaolin, lanolin, mineral oil, sodium bicarbonate, zinc acetate, zinc carbonate, zinc oxide, aluminum acetate, aluminum sulfate, witch hazel, selenium sulfide, pyrithione zinc, selenium sulfide, coal tar, hydroquinone, benzocaine, lidocaine.

### Manufacturing

The compositions for drug delivery in accordance with the present invention may be manufactured using any suitable method and conditions.

In embodiments, obtaining the composition for drug delivery comprises mixing the API into a stable homogeneous aqueous comprising insoluble and/or semi-soluble particles.

In embodiments, manufacturing the compositions for drug delivery comprises mechanically processing together the API with biopolymer(s) under high-shearing conditions and/or under high mechanical energy to obtain a stable homogeneous aqueous composition as defined herein, the composition comprising insoluble and/or semi-soluble particles, with the API(s) dispersed therein. The Exemplification section hereinafter provides some examples of such manufacturing.

The compositions for drug delivery in accordance with the present invention are preferably stable. Stability at least means that the biopolymer (e.g., fibers, spherical bodies) or any other component of the compositions, does not settle at the bottom. In embodiments the insoluble and/or semi-soluble biopolymer(s) remains in suspension for at least 1 week, or at least 1 month, or at least 6 months, or at least 12 months, or at least 18 months, or at least two years, or at least three years or more.

Preferably, the compositions for drug delivery in accordance with the present invention also provide a stable environment to the API(s) comprised in the composition. Those skilled in the art can readily identify and provide such a stable environment to the API, for instance by selecting proper biopolymer or combinations thereof, identifying proper concentrations or ratios for the biopolymer(s) and/or API, by adding additional compounds (e.g., stabilizer, pH modifier, binding agent, salt, crosslinker, co-solvent, softener etc.).

The compositions for drug delivery in accordance with the present invention may be formulated as a paste, an ointment, a cream, a lotion, a gel or a milk. In embodiments desired formulations are obtained by obtaining compositions having a desired viscosity. In embodiments the viscosity of the compositions/suspensions can be altered by varying the high-shearing conditions and/or mechanical energy to which the biopolymer(s) are submitted. In embodiments the stable homogeneous suspension comprises a viscosity of about 25 mPa to about 85 000 mPa. Table 1 below provides non-limiting examples of desirable viscosity for the compositions/suspensions in accordance with the present invention.

TABLE 1
Examples of desired viscosities

| Desired Consistency | Exemplary viscosity (mPa · s) |
|---|---|
| paste | about 40000 to about 300000 or higher |
| ointment | about 20000 to about 50000 |
| cream | about 1500 to about 30000 |
| lotion | about 800 to about 4000 |
| gel | about 1000 to about 40000 |
| milk | about 20 to about 2000 |

In embodiments, the viscosity the compositions for drug delivery in accordance with the present invention may be varied by selecting accordingly the biopolymer(s) comprised in the composition and/or the API.

The viscosity the compositions for drug delivery in accordance with the invention may also be varied in accordance with the ratios or concentration of each of the compound entering into the composition, in addition to the biopolymer. In embodiments the weight ratio of biopolymers: API is of about 0.1:20 to about 10:20, or about 0.5:20 to about 3:20, or about 0.75:20, or about 1.0:20, 1.25:20. or about 1.5:20.

In embodiments the compositions for drug delivery in accordance with the invention comprises about 0.01-10% w/w of biopolymer, or 0.01-5% w/w of biopolymer, or 0.01-2% w/w of biopolymer, or 0.01-1% w/w of biopolymer. In embodiments the compositions in accordance with the invention comprises about 0.01% w/w of biopolymer, or about 0.05% w/w of biopolymer, or about 0.1% w/w of biopolymer, or about 0.25% w/w of biopolymer, or about 0.5% w/w of biopolymer, or about 0.75% w/w of biopolymer, or about 1% w/w of biopolymer, or about 1.5% w/w of biopolymer, or about 2.5% w/w of biopolymer, or about 5% w/w of biopolymer.

The compositions for drug delivery in accordance with the invention may also be formulated as stable emulsions comprising fatty acids (e.g. C10-C22 fatty acids), an oil and/or a wax, and/or comprising N-Acetyl Glucosamine, and/or emulsifiers and preservatives, and/or comprising additives, including, but not limited to, preservatives, stabilizers and emulsifiers. In embodiments the additive is selected from Cetyl alcohol, Glyceryl stearate, Soy butter, PC90, Tara Gum, PSC3, PEG, Guar, Xanthan gum, Agarose, Sodium Hyaluronate, Tween 80™ and Glycerol. The additive(s) may be added prior, during and/or after combination of the biopolymer with the API.

The drug delivery compositions of the invention may also be provided in a dried form and formulated as tablets, capsules, pellets, granules, films, transdermal patches, transplants, scaffolds, suppositories, etc.

### Methods of Delivery and Related Uses

The compositions with the present invention may find numerous applications in the delivery of compounds of interest (e.g., API) to subjects in need thereof.

In aspects, the invention concerns a method for delivery of an active pharmaceutical ingredient (API) to a subject in need thereof. In one embodiment the method comprises providing a composition as defined herein and administering the composition to the subject. The composition may be administered using any suitable route allowing for the delivery and release of API at a desired site.

For instance, formulations may be prepared so as to provide a composition formulated in a form which is suitable for any route of administration such as topical administration, oral administration, sublingual, buccal, rectal, vaginal, ocular, otic, nasal, cutaneous, and transdermal administration. In preferred embodiments the route of administration is topical or oral.

According to one particular aspect, the administration is topical and the invention concerns a method for topical delivery. In one embodiment, the method comprises providing an aqueous composition comprising a biopolymer and at least one API and applying topically the composition on the body of the subject.

The compositions may be applied on any desired surface of the subject's body including, but not limited to, its hands, arms, legs, feet, torso, breast, face, neck, scalp, etc. In embodiments, the composition is applied on an area of the skin that comprises a wound, a scar, a wrinkle and or any other type of damage. For topical uses, the composition may be formulated as a paste, an ointment, a cream, a lotion, a gel or a milk.

According to one particular aspect, the administration is oral and the invention concerns a method for topical delivery. In one embodiment, the method comprises (i) providing a mixture comprising an API mixed in a biopolymer composition; (ii) drying the mixture to obtain a dried product; and (iii) orally administering the dried product to the subject. For oral uses, the dried product may take the form of tablets, capsules, pellets, granules, etc. For other modes of administration, the dried product may take the form offilms, transdermal patches, transplants, scaffolds, suppositories, etc.

Those skilled in the art will recognize, or be able to ascertain, using no more than routine experimentation, numerous equivalents to the specific procedures, embodiments, claims, and examples described herein. Such equivalents are considered to be within the scope of this invention, and covered by the claims appended hereto. The invention is further illustrated by the following examples, which should not be construed as further or specifically limiting.

### EXAMPLES

Experiments were carried out to demonstrate the release of small pharmaceutical molecules or API from biopolymer compositions in accordance with the present invention.

Briefly biopolymer suspensions were produced with a known quantity of API added to the suspensions. The suspensions with API were then dried as pellets. These pellets were then added to a known volume of water and aliquots of the water were removed periodically to determine the rate of release of the API. The API concentration was measured using UV/VIS spectroscopy.

### Example 1: Acetaminophen in Chitin

An acetaminophen-biopolymer suspension was generated by milling together 21.88 g chitin suspension (1.05:20) with 0.50 g of acetaminophen at 670 RPM with fifty units of 10 mm ball using the 10+1 Alt method, where it is milled for ten minutes followed by a pause for one minute then milling for ten minutes in the opposite direction for a total of 2 hours. The concentration of acetaminophen in the aqueous suspension so obtained was 2.23% w/w. The chitin suspension was produced with a 1.5 L Supermill Plus™ with 1.4-1.7 mm zirconia beads in a 20 L batch by milling with the general milling conditions of 2400 FPM (feet per minute) rotation speed with a pump flow rate of 7.3 GPH (gallons per hour) using 982 mL of 1.4-1.7 mm zirconia beads, where 20 liters of slurry were processed in a 5% solids content (1.05:20).

The following table shows the viscosity of the final aqueous suspension:

| Shear Rate (Hz) | Viscosity (mPa · s) |
|---|---|
| 0.14 | 181500 |
| 0.28 | 81170 |
| 0.56 | 38330 |
| 1.4 | 15350 |
| 2.24 | 9042 |
| 2.8 | 6983 |

The sample was dried for 10 hours in air and 3 hours in the oven at 50° C. The acetaminophen concentration in dried materials was 29.58% w/w.

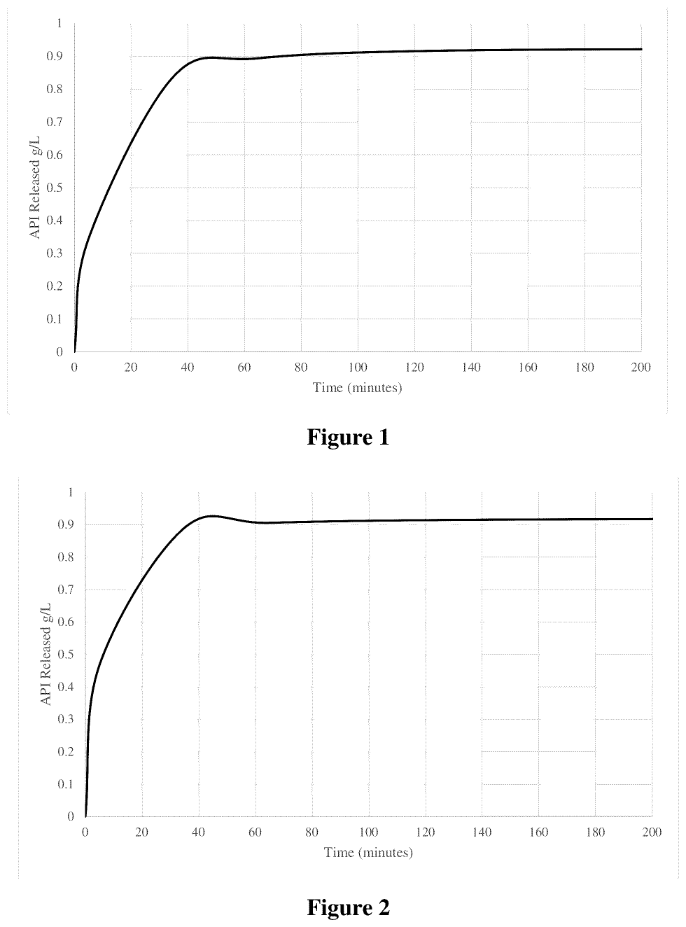

The acetaminophen-biopolymer suspension could successfully release acetaminophen as shown in FIG. 1. This figure represents release of acetaminophen into water from the dried acetaminophen doped chitin suspension, where the concentration of acetaminophen, in g/L, is plotted against time, in minutes. As can be seen, the largest portion of acetaminophen was released in the first 35-50 minutes followed by slow release for the next 23 hours, which suggests a zero-order controlled release.

### Example 2: Acetaminophen in Chitosan

An acetaminophen-biopolymer suspension was generated by milling together 22.64 g chitosan suspension (1.05:20) with 0.50 g of acetaminophen at 670 RPM with fifty units of 10 mm ball using the 10+1 Alt method, where it is milled for ten minutes followed by a pause for one minute then milling for ten minutes in the opposite direction for a total of 2 hours. The concentration of acetaminophen in the aqueous suspension so obtained was 2.16% w/w. The chitosan suspension was produced with a 1.5 L Supermill Plus™ with 1.4-1.7 mm zirconia beads in a 20 L batch by milling with the general milling conditions of 2400 FPM (feet per minute) rotation speed with a pump flow rate of 7.3 GPH (gallons per hour) using 982 mL of 1.4-1.7 mm zirconia beads, where 20 liters of slurry were processed in a 5% solids content (1.05:20).

The following table shows the viscosity of the final aqueous suspension:

| Shear Rate (Hz) | Viscosity (mPa · s) |
|---|---|
| 0.28 | 471500 |
| 0.56 | 208300 |
| 1.4 | 64450 |
| 2.24 | 29760 |
| 2.8 | 21710 |

The sample was dried for 10 hours in air and 3 hours in the oven at 50° C. The acetaminophen concentration in dried materials was 25.23% w/w.

The acetaminophen-biopolymer suspension could successfully release acetaminophen as shown in FIG. 2. This figure represents release of acetaminophen into water from the dried acetaminophen doped chitosan suspension, where the concentration of acetaminophen, in g/L, is plotted against time, in minutes. As can be seen, the largest portion of acetaminophen was released in the first 35-50 minutes followed by slow release for the next 23 hours, which suggests a zero-order controlled release.

### Example 3: Acetylsalicylic Acid in Chitosan

An acetylsalicylic acid-biopolymer suspension was generated by milling together 22.09 g chitosan with 0.50 g of acetyl salicylic acid at 670 RPM with fifty units of 10 mm ball using the 10+1 Alt method, where it is milled for ten minutes followed by a pause for one minute then milling for ten minutes in the opposite direction for a total of 2 hours. The concentration of acetyl salicylic acid in the aqueous suspension so obtained 2.21% w/w. The chitosan suspension was produced with a 1.5 L Supermill Plus™ with 1.4-1.7 mm zirconia beads in a 20 L batch by milling with the general milling conditions of 2400 FPM (feet per minute) rotation speed with a pump flow rate of 7.3 GPH (gallons per hour) using 982 mL of 1.4-1.7 mm zirconia beads, where 20 liters of slurry were processed in a 5% solids content (1.05:20).

The following table shows the viscosity of the final aqueous suspension:

| Shear Rate (Hz) | Viscosity (mPa · s) |
|---|---|
| 0.14 | 210300 |
| 0.28 | 99750 |
| 0.56 | 53040 |
| 1.4 | 23150 |
| 2.24 | 16120 |
| 2.8 | 13310 |

The sample was dried for 10 hours in air and 3 hours in the oven at 50° C. The acetylsalicylic acid concentration in dried materials was 34.99% w/w.

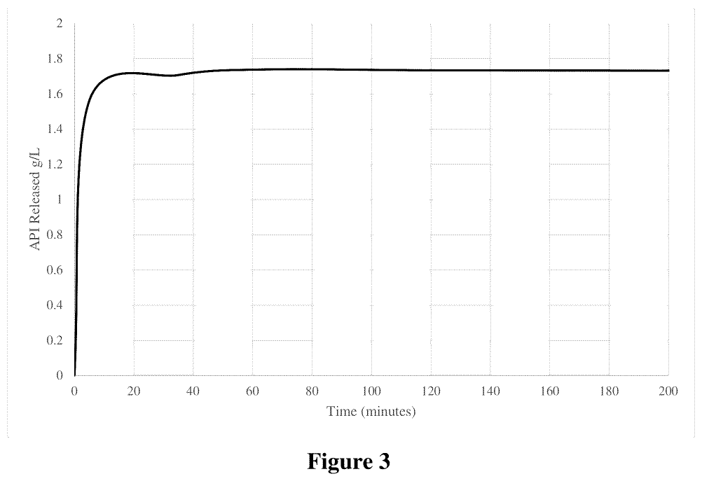

The acetylsalicylic acid-biopolymer suspension could successfully release acetaminophen as shown in FIG. 3. This figure represents release of acetylsalicylic acid into water from the dried acetylsalicylic acid doped chitosan suspension, where the concentration of acetylsalicylic acid, in g/L, is plotted against time, in minutes. As can be seen, the largest portion of acetylsalicylic acid is released in the first 35-50 minutes followed by slow release for the next 23 hours, which suggests a zero-order controlled release.

### DISCUSSION

The present examples demonstrate that biopolymer compositions in accordance with the present invention can successfully be used for drug delivery since acetaminophen and acetyl salicylic acid were both added to biopolymer suspensions and later released from the dried suspensions.

The present examples also demonstrate that the present biopolymer suspensions can be helpful in controlling the release of different drugs since acetaminophen showed a slower release from the dry biopolymer matrix than did the acetyl salicylic acid.

In this regard, without wishing being bound by theory, the particle size of the polymer may have an effect on the release rate. Accordingly, it may be possible to control the rate of release of a desired API by modifying the polymer suspension particles size.

Headings are included herein for reference and to aid in locating certain sections. These headings are not intended to limit the scope of the concepts described therein, and these concepts may have applicability in other sections throughout the entire specification. Thus, the present invention is not intended to be limited to the embodiments shown herein but is to be accorded the widest scope consistent with the principles and novel features disclosed herein.

The singular forms “a”, “an” and “the” include corresponding plural references unless the context clearly dictates otherwise. Thus, for example, reference to “a biopolymer” includes one or more of such biopolymers and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and such.

It is understood that the examples and embodiments described herein are for illustrative purposes only and that various modifications or changes in light thereof will be suggested to persons skilled in the art and are to be included within the present invention and scope of the appended claims.

1. A composition for delivery of an active pharmaceutical ingredient (API), comprising: a biopolymer, and at least one API, wherein the API and the biopolymer are stably dispersed to form together said composition.

2. (canceled)

3. The composition according to claim 1, wherein the composition comprises biopolymer molecules that have been mechanically processed into a stable homogeneous aqueous suspension.

4. The composition according to claim 1, wherein the composition comprises an homogeneous aqueous suspension of insoluble and/or semi-soluble biopolymer particles.

5. The composition according to claim 4, wherein the stable homogeneous aqueous suspension comprises insoluble and/or semi-soluble biopolymer particles stably dispersed within a polar solvent.

6. The composition according to claim 1, wherein the biopolymer consists of a stable homogeneous aqueous suspension composed of insoluble and/or semi-soluble biopolymer particles.

7. The composition according to claim 1, wherein the biopolymer is selected from the group consisting of chitin, chitosan, cellulose, hemicellulose, lignin, amylose, actin, fibrin, collagen, silk, fibroin, keratin, wool, alginic acid and mixtures thereof.

8. (canceled)

9. The composition according to claim 1, wherein said composition is formulated as a paste, an ointment, a cream, a lotion, a gel or a milk.

10. (canceled)

11. The composition according to claim 1, wherein said composition is formulated as a tablet, a capsule, a pellet, a granule, a transdermal patch, a transplant, a scaffold, or a suppository.

12. The composition according to claim 1, wherein said API is a pharmaceutical product selected from the group consisting of analgesics, anesthetics, anti-addiction agents, anti-bacterials, anticonvulsants, antidementia agents, antidepressants, antiemetics, antifungals, antigout agents, anti-inflammatoires, antimigraine agents, anti-myasthenic agents, anti-mycobacterials, antineoplastics, anti-obesity agents, anti-parasitics, anti-parkinson agents, antipsychotics, anti-spasticity agents, antivirals, anxiolytics, bipolar agents, blood glucose regulators, blood products, cardiovascular agents, central nervous system agents, contraceptives, dental and oral agents, dermatological agents, electrolytes, minerals, metals, vitamins, gastrointestinal agents, genitourinary agents, hormonal agents (adrenal), hormonal agents (pituitary), hormonal agents (prostaglandins), hormonal agents (sex hormones), hormonal agents (thyroid), hormone suppressant (adrenal), hormone suppressant (pituitary), hormone suppressant (thyroid), immunological agents, infertility agents, inflammatory bowel disease agents, metabolic bone disease agents, ophthalmic agents, otic agents, respiratory tract agents, sexual disorder agents, skeletal muscle relaxants, sleep disorder agents, and mixtures thereof.

13. The composition according to claim 12, wherein said pharmaceutical product is selected from the group consisting of acetaminophen, acetyl salicylic acid, aciclovir (including Zovirax™), acrivastine, adalimumab, alendronic acid, allopurinol, alogliptin, amitriptyline for depression, amitriptyline for pain and migraine, amlodipine, amoxicillin, anastrozole, apixaban, Aspirin™, atenolol, atorvastatin, azathioprine, azithromycin, baclofen, beclometasone inhalers, beclometasone nasal spray, beclometasone skin creams, bendroflumethiazide, benzoyl peroxide, benzydamine, betahistine, betamethasone for eyes, ears and nose, betamethasone for skin, bimatoprost, bisacodyl, bisoprolol, brinzolamide, budesonide inhalers, budesonide nasal spray, budesonide rectal foam and enemas, bumetanide, buprenorphine for pain, buscopan (hyoscine butylbromide), candesartan, carbamazepine, carbimazole, carbocisteine, carmellose sodium, carvedilol, cefalexin, cetirizine, Champix™ (varenicline), chloramphenicol, chlorhexidine, chlorphenamine (Piriton™), cinnarizine, ciprofloxacin, citalopram, clarithromycin, clobetasol, clobetasone, clonazepam, clopidogrel, clotrimazole, clotrimazole for thrush (Canesten™), co-amoxiclav, co-beneldopa, co-careldopa, co-codamol for adults, co-codamol for children, co-codaprin (aspirin and codeine), co-dydramol, codeine, colchicine, cyanocobalamin, cyclizine, dabigatran, dapagliflozin, dexamethasone tablets and liquid, diazepam, diclofenac, digoxin, dihydrocodeine, diltiazem, diphenhydramine, dipyridamole, docusate, domperidone, donepezil, dosulepin, doxazosin, doxycycline, duloxetine, edoxaban, empagliflozin, enalapril, eplerenone, erythromycin, escitalopram, esomeprazole, ezetimibe, felodipine, fentanyl, ferrous fumarate, ferrous sulfate, fexofenadine, finasteride, flucloxacillin, fluconazole, fluoxetine (Prozac™), fluticasone inhalers, fluticasone nasal spray and drops, fluticasone skin creams, folic acid, furosemide, fusidic acid, fybogel (ispaghula husk), gabapentin, GavisconT (alginic acid), gliclazide, glimepiride, glyceryl trinitrate (gtn), heparinoid, hydrocortisone, hydrocortisone buccal tablets, hydrocortisone for piles and itchy bottom, hydrocortisone for skin, hydrocortisone injections, hydrocortisone rectal foam, hydrocortisone tablets, hydroxocobalamin, hyoscine hydrobromide (kwells and joy-rides), ibuprofen and codeine, ibuprofen for adults (Nurofen™), ibuprofen for children, indapamide, irbesartan, isosorbide mononitrate and isosorbide dinitrate, isotretinoin capsules (Roaccutane™), isotretinoin gel (Isotrex™), ketoconazole, labetalol, lactulose, lamotrigine, lansoprazole, latanoprost, lercanidipine, letrozole, levetiracetam, levothyroxine, lidocaine for mouth and throat, lidocaine for piles and itchy bottom, lidocaine skin cream, linagliptin, lisinopril, lithium, loperamide, loratadine (Clarityn™), lorazepam, losartan, low-dose aspirin, lymecycline, macrogol, mebendazole, mebeverine, melatonin, mesalazine, metformin, methadone, methotrexate, methylphenidate for adults, methylphenidate for children, metoclopramide, metoprolol, metronidazole, mirabegron, mirtazapine, molnupiravir (Lagevrio™), mometasone for skin, mometasone inhalers, mometasone nasal spray, montelukast, morphine, naproxen, nefopam, nicorandil, nifedipine, nitrofurantoin, nortriptyline, nystatin, olanzapine, olmesartan, omeprazole, oxybutynin, oxycodone, pantoprazole, paracetamol for adults, paracetamol for children (including calpol), paroxetine, paxlovid, peppermint oil, Pepto-Bismol™, perindopril, phenoxymethylpenicillin, phenytoin, pioglitazone, pravastatin, prednisolone tablets and liquid, pregabalin, prochlorperazine, promethazine (Phenergan™), propranolol, pseudoephedrine (including Sudafed™), rabeprazole, ramipril, ranitidine, remdesivir (VekluryT), risedronate, risperidone, rivaroxaban, ropinirole, rosuvastatin, salbutamol inhaler, saxagliptin, senna, sertraline, sildenafil (Viagra™), simeticone, simvastatin, sitagliptin, sodium valproate, solifenacin, sotalol, sotrovimab (Xevudy™), sulfasalazine, sumatriptan, tadalafil, tamsulosin, temazepam, terbinafine, thiamine (Vitamin B1), ticagrelor, tolterodine, topiramate, tramadol, tranexamic acid, trazodone, trimethoprim, valproic acid, valsartan, varenicline (Champix™) venlafaxine, verapamil, zolpidem, zopiclone, and mixtures thereof.

14. The composition according to claim 1, wherein said API is a cosmetic product selected from the group consisting of a compound for moisturizing the skin, a compound for improving skin appearance, a compound for protecting from environmental damages, a compound for fighting the effects of aging, a compound for removing wrinkles and/or sunspots, and/or a compound for the prevention of any of the following skin conditions: acne, acne/folliculitis, acne scars, aging skin, blemishes, broken blood vessels, brown spots or other discolored skin, cellulite, creases around the mouth or nose, deflated or sinking around cheeks, temples, lips and eyes, dull and discolored skin, excessive facial or body hair, enlarged pores, fatigued or tired appearance, fine lines and creases, flaking and cracking skin, flushed appearance, freckles, furrows or crinkles in the forehead, hair loss, hair removal, hirsutism and hypertrichosis, hyperpigmentation, melasma, rosacea, sagging or loss of volume, scarring of skin, skin cancer, spider veins in the legs and face, sun damaged skin, sunspots, tattoo removal, textural and tone changes, tiny lines near the eyes or mouth, visible facial blood vessels, wrinkles and fine lines, and mixtures thereof.

15. The composition according to claim 1, wherein said API is a cosmetic compound is selected from the group consisting of: antioxidants (e.g. resveratrol, vitamin C), alpha hydroxy acid (AHA), hyaluronic acid, retinoids (e.g., retinol), vitamins (e.g., vitamin A, vitamin E), resorcinol, benzoyl peroxide, salicylic acid, ceramides, niacinamide, sulfur, sodium fluoride, sodium monophosphate, clioquinol, haloprogin, miconazole nitrate, povidone-iodine, tolnaftate, undecylenic acid, calcium undecylenate, copper undecylenate, zinc undecylenate, clotrimazole bacitracin, chlortetracycline, neomycin sulfate, tetracycline hydrochloride, aluminum chloride, aluminum chlorohydrate, aluminum chlorohydrex polyethylene glycol, aluminum chlorohydrex propylene glycol, aluminum dichlorohydrate, aluminum dichlorohydrex polyethylene glycol, aluminum dichlorohydrex propylene glycol, aluminum sesquichlorohydrate, aluminum sesquichlorohydrex polyethylene glycol, aluminum sesquichlorohydrex propylene glycol, aluminum zirconium octachlorohydrate, aluminum zirconium octachlorohydrex gly, aluminum zirconium pentachlorohydrate, aluminum zirconium pentachlorohydrex gly, aluminum zirconium tetrachlorohydrate, aluminum zirconium tetrachlorohydrex gly, aluminum zirconium trichlorohydrate, aluminum zirconium trichlorohydrex gly, allantoin, aluminum hydroxide gel, calamine, cocoa butter, cod liver oil, dimethicone, glycerin, kaolin, lanolin, mineral oil, sodium bicarbonate, zinc acetate, zinc carbonate, zinc oxide, aluminum acetate, aluminum sulfate, witch hazel, selenium sulfide, pyrithione zinc, selenium sulfide, coal tar, hydroquinone, benzocaine, lidocaine, and mixtures thereof.

16. A method for delivery of an active pharmaceutical ingredient (API) to a subject in need thereof, comprising: (i) providing a composition for delivery as defined in claim 1; and (ii) administering said composition to the subject.

17. The method of claim 16, wherein said composition is administered by a route of administration selected from the group consisting of topical, oral administration, sublingual, buccal, rectal, vaginal, ocular, otic, nasal, cutaneous, and transdermal administration.

18. The method of claim 16, wherein said composition is formulated as a paste, an ointment, a cream, a lotion, a gel or a milk.

19. The method of claim 16, wherein said composition is as a tablet, a capsule, a pellet, a granule, a film, a transdermal patch, a transplant, a scaffold, or a suppository.

20. (canceled)

21. The method of claim 16, wherein the composition is applied on a surface of the subject's body selected from the group consisting of the hands, arms, legs, feet, torso, breast, face, neck and scalp.

22. (canceled)

23. The method of claim 21, wherein the composition is applied as a transdermal patch.

24. (canceled)

25. (canceled)

26. A method for manufacturing a composition for delivery as defined in claim 1, the method comprising mechanically processing together the API with biopolymer(s) under high-shearing conditions and/or under high mechanical energy to obtain a stable homogeneous aqueous composition.

27. The method of claim 26, wherein said mechanically processing is carried out until a stable homogeneous aqueous suspension is obtained, said suspension comprising insoluble and/or semi-soluble particles with the API dispersed therein.
